(19) United States
(12) Patent Application Publication
(10) Pub. No.: US 2015/0313995 A1
(43) Pub. Date: Nov. 5, 2015

(54) Magnetic Retention of Regenerative Cells for Wound Repair

(71) Applicant: MEDIVATION TECHNOLOGIES, INC., San Francisco, CA (US)

(72) Inventor: David Hung, San Francisco, CA (US)

(21) Appl. No.: 14/651,879

(22) PCT Filed: Dec. 12, 2013
(86) PCT No.: PCT/US13/74577

§ 371 Date: Jun. 12, 2015

Related U.S. Application Data
(60) Provisional application No. 61/800,169, filed on Mar. 15, 2013.
(60) Provisional application No. 61/737,446, filed on Dec. 14, 2012.

(51) Int. Cl. A61K 41/00; A61N 2/02; A61K 9/16; A61K 47/48; A61K 35/32; A61K 35/28

(52) U.S. Cl. CPC A61K 41/0052; A61K 35/32; A61K 35/28; A61K 9/16; A61K 47/48853; A61N 2/02

(57) ABSTRACT

Methods are disclosed for promoting wound repair, by magnetically retaining or confining stem cells, or other cells capable of generating the desired tissue, in a target region (e.g., a wound) where tissue regrowth is needed. Also disclosed are magnetic particles and magnetically inducible particles for these methods, as well as sheets comprising magnetic materials.

### CROSS-REFERENCE TO RELATED APPLICATIONS

This application claims the benefit of priority of U.S. Application Ser. No. 61/800,169, filed Mar. 15, 2013 and U.S. Application Ser. No. 61/737,446, filed Dec. 14, 2012, the disclosures of which are hereby incorporated by reference in their entireties.

### FIELD OF THE INVENTION

The present invention relates to the use of a plurality of magnetic or magnetically inducible particles in the body, in order to magnetically retain stem cells or other regenerative cells at the site of a wound, thereby promoting its repair.

### BACKGROUND

Tissue damage can result from a number of conditions, including disease, surgery, exposure to harmful substances, injury, and aging. Wounds can occur in any tissue of the body, including tissues of joint surfaces (e.g., articular cartilage), bone tissues (e.g., periosteum), connective tissues (e.g., tendons and ligaments), spinal cord tissue, and tissues of organs such as the heart, pancreas, kidney, liver, eye, brain, gallbladder, prostate, breast, intestines, skeletal muscle, and lung. The treatment of various forms of tissue damage is complicated by the different cell types involved and, in many cases, the difficulty in accessing the wound site.

Known treatments for internal tissue wounds involve the implantation of artificial devices that facilitate repair and regeneration. For example, U.S. Pat. No. 5,981,825 and U.S. Pat. No. 5,716,404 disclose anatomically specific and bioresorbable implant devices for facilitating the healing of voids in bone, cartilage, and soft tissue. In addition, proposed tissue engineering techniques employ a combination of artificial and biological approaches to facilitate the body's own effort to heal or generate new tissues. For example, U.S. Pat. No. 5,885,829 describes such methods for use in regenerating dental and oral tissues from viable cells, using ex vivo culture on a structural matrix. The use of cultured cells and/or extracellular matrix, alone or combined with artificial devices, is also known for tissue construction and generation in other applications. For example, U.S. Pat. No. 5,919,702 describes the isolation and use of pre-chondrocytes from the umbilical cord, which give rise to chondrocytes that produce cartilage for implantation and repair of joint tissue deficiencies. U.S. Pat. No. 5,830,708 describes the production of naturally secreted human extracellular matrix material that is useful for the repair of soft tissue and skin defects.

The implantation of tissue-specific regenerating cells, or cells having the potential to differentiate to reconstruct the desired tissue, has been considered a promising alternative for wound treatment, compared to known surgical procedures. Effective methods based on this approach would be particularly desirable in the case of tissues such as articular cartilage that have only a limited potential for self-healing. Moreover, in conventional joint treatment methods, which include drilling, abrasion, microfracture, and distraction, the defects are repaired by fibrous tissues rather than the lost hyaline cartilage.

Recent developments in techniques for the isolation, purification, and culturing of stem cells have prompted considerable research into their use for tissue wound repair and regeneration procedures. Stem cells can differentiate into different cell lineages due to their self-renewing and clonogenic capabilities. They are thus considered “generic” cells that are capable of growing into specialized cells, e.g., brain cells, muscle cells, bone cells, or blood cells that perform specific body functions. Embryonic stem cells have the capability to differentiate into any terminally differentiated cell in the body. Adult stem cells, originally thought to have more limited capability, may be programmed under specific signals to differentiate into organ-specific cells having a phenotype distinct from that of the precursor. Stem cells can be administered via systemic intravascular route or a direct local implantation, such as performed presently to repair infracted myocardium and in spinal cord injuries. Arthroscopic implantation may be performed to incorporate stem cells into articular cartilage of a damaged joint or other internal wound site.

There is a particular interest in the more “committed” stem cells, capable of becoming many types of cells, but not all types (as in the case of embryonic stem cells). Mesenchymal stem cells (MSCs) are representative of a line of such “pluripotent” formative cell types. MSCs are capable of differentiating into any of the specific types of mesenchymal or connective tissues, including adipose, osseous, cartilaginous, elastic, and fibrous connective tissues, depending upon various influences from bioactive factors (e.g., cytokines). MSCs are found, among other areas, in the bone marrow, blood, dermis, and periosteum. From these sites, they can migrate to a remote site of injury and regenerate the required tissue type. Synovium-derived mesenchymal stem cells (SDMSCs) are particular members of the MSC family that have a high potential for both proliferation and differentiation. Synovial tissues are easily harvested and may be collected from any joint without damaging articular cartilage.

MSCs are identified by specific cell surface markers, and these markers can in turn allow the isolation of MSCs from other cell types through specific antigen-monoclonal antibody recognition. A homogeneous MSC composition may be obtained, for example, by positive selection of adherent marrow or periosteal cells that are free of markers associated with either hematopoietic cell or differentiated mesenchymal cells. The isolated MSC population displays epitopic characteristics specific to this cell line, which has the ability to grow in culture without differentiating (e.g., through mitotic expansion in a specific medium), as well as differentiate into specific mesenchymal lineages, for example when induced in vitro or placed in vivo at the site of damaged tissue. Human MSCs have the ability to differentiate into osteoblasts and chondrocytes, which form a wide variety of mesenchymal tissues, such as hyaline cartilage, as well as tendon, ligament, and dermis tissues. This potential is retained after isolation and even for several population expansions in culture. Techniques for isolating MSCs based on antibody interactions with specific cell surface antigens are described, for example, in Motoyama et al., J. BIOMED. MAT. RES., Part A (2009): 196-204. The specific technique discussed in this reference utilizes magnetically tagged MSCs.

The use of MSCs, following isolation and multiplication in cell culture, for treating skeletal and other connective tissue disorders is described, for example, in U.S. Pat. No. 5,226,914. U.S. Pat. No. 5,906,934 describes the administration of certain MSCs seeded in a polymeric carrier suitable for proliferation and differentiation of the cells into articular cartilage or subchondral bone. Other applications of MSCs in the treatment of joint tissues are described, for example, in the patent documents U.S. Pat. No. 7,971,592; U.S. Pat. No. 5,908,784; U.S. Pat. No. 5,837,539; U.S. Pat. No. 5,811,094; and US 2008/0118569; as well as in Singh, BMC MEDICINE (2012), Vol. 10(44): 1-5; and Hori et al., J. ORTHOPAED. RES. (April 2011): 531-8. Some of these references employ magnetically tagged MSCs as described in Montoyama, cited above.

The treatment of many types of wounds or lesions in the body may be possible through the isolation, purification, and multiplication of MSCs, followed by activating these cells in vivo to cause their differentiation into the specific mesenchymal cell types desired. In the case of regenerating articular cartilage of joint tissues, the implantation of MSCs can be accomplished with far less invasive procedures, for example using an arthroscope or syringe, relative to conventional joint replacement surgery. In addition, MSCs offer the possibility for regrowth of native hyaline cartilage that is more desirable than fibrous tissue.

However, the practical use of MSCs, as well as other cells that may be used for tissue regeneration and repair, is complicated by the difficulty in uniformly distributing and retaining these cells, along with any necessary growth factors, at the site of the wound in sufficient concentration and for a sufficient time to promote effective healing. The efficacy of a treatment regimen can become seriously compromised if the location of the implanted cells is not sufficiently controlled following injection, for example if MSCs migrate rapidly from the wound site to the joint space generally and beyond. In order to promote cell adherency in practice, current treatment regimens can require patients to minimize their movement and completely eliminate weight bearing activities for several days following the injection of stem cells. Limits on patients' activities may extend as long as several weeks or even a few months thereafter. For these reasons, improvements in the ability to confine regenerative cells to where they are needed, at least for an initial phase of cell engraftment, would greatly benefit the present state of the art.

### SUMMARY OF THE INVENTION

Aspects of the present invention are associated with the discovery of methods for promoting wound repair, by magnetically isolating or confining stem cells, or other cells capable of generating the desired tissue, in a target region (e.g., a wound) where tissue regrowth is needed. The cells are complexed with a magnetizable substrate, meaning that the substrate itself does not generate a magnetic field, but is magnetic in the presence of one and is therefore attracted to a magnet. A representative magnetizable substrate comprises an underlying polymer base (e.g., a styrene co-polymer) that is coated with a thin layer of ferromagnetic material such as an iron oxide compound. The substrate may be complexed through antigen-antibody interaction or binding with a surface marker that is specific to the desired cell type (e.g., the CD44 glycoprotein marker of mesenchymal stem cells). The antibody can therefore serve as a type of cell surface recognition ligand that is bonded to the magnetizable substrate. Bonding can occur using a suitable chemical linkage, such as a peptide linkage formed between an amine group or a carboxyl group of the cell surface recognition ligand and a carboxyl group or amine group that is used to derivatize the surface of the magnetizable substrate for covalent amide bonding.

Advantageously, a plurality of magnetic particles is implanted proximate the target region, allowing for customization of a magnetic field to the geometry of the wound. For example, a cluster of magnetic particles implanted directly beneath the damaged tissue may provide an essentially uniform magnetic field, in terms of both strength and direction, about the wound surface. Variation in the depth of the individually implanted magnetic particles may be tailored to the wound “topography,” or its three-dimensional shape features. In other embodiments, a relatively greater magnetic field strength may be desired at the wound periphery relative to a central area, in order to strongly inhibit cell migration out of the target region, but allow greater cell mobility within this region. In one example of a target region of damaged articular cartilage, the magnetic particles may be implanted immediately below this region in the subchondral bone. In this case, it may be desirable to further adapt these particles for bone implantation by attaching (e.g., through covalent bonding) a bone-compatible polymer to their surfaces. The bone-compatible polymer, which may also be biodegradable, aids in the fixation or stabilization of the magnetic particles within the bone structure. That is, the bone-compatible polymer may act as a “barb” or “anchor” for the magnetic particles. Those skilled in the art will appreciate from the present disclosure that other tissue-compatible substituents, including polymers, may be attached to the magnetic particles to improve the stability of their implantation into other tissues.

The use of a plurality of magnetic particles, preferably of a size that is small relative to that of the wound, provides several advantages over the use of a single magnet that must necessarily be made larger and have a greater overall magnetic field strength, in order to ensure the presence of at least a minimum strength in all areas of the wound. These attributes can lead to undesirable piling or “stacking” of cells in an area of high magnetic field strength (e.g., in a central area), thereby disrupting initial, uniform cell growth in a monolayer about the target region and/or interfering with proper engraftment. In fact, the existence of an excessive magnetic field alone can result in abnormal cell growth, proliferation, differentiation, and engraftment. In contrast, magnetic particles can provide small, locally acting magnetic fields that interfere with each other to a minimal extent or not at all. In other embodiments, magnetic field interference between adjacent and/or surrounding magnetic particles may be beneficial, in order to create a combined magnetic field having desirable characteristics of magnitude and direction about the surface of a wound. Cells positioned between magnetic particles will be directed along magnetic lines of force. Depending on the positioning of the magnetic particles, these magnetic lines of force can be made to overlap with the target region for regenerative (wound repair) therapy. In specific embodiments, the magnetic particles are in the form of spheres having an average diameter of less than about 1 millimeter (mm) and a magnetization (magnetic moment per unit volume) of less than about 10,000 amperes/meter (A/m), less than about 5,000 A/m, less than about 1,000 A/m, or even less than about 500 A/m.

A magnetic field for isolating magnetized cells may be alternatively generated by implanting magnetically inducible particles, rather than permanently magnetic particles, or otherwise a combination of such magnetic and magnetically inducible particles. This allows for variation of the magnetic field strength in the target region, in order provide customization in both time and space, for example by varying an inducing electric current. Such a current may surround, or be external to, magnetically inducible particle(s), as in the case of a current passing through a coil that is external to the wound site and normally outside the patient's body. According to one embodiment, the magnetically inducible particles may deliver a pulsed magnetic field that can allow cells to uniformly seed a wound in some cases. For example, during times of a pulsed magnetic field delivery when the magnetic field is “on,” the cells may be magnetically attracted to the target region, whereas when the magnetic field is “off,” the cells may be allowed to assume a more desirable distribution (e.g., more in the form of a monolayer, as opposed to an agglomerated cell mass near the vicinity of a magnetically inducible particle), thereby beneficially providing wider and more uniform cell coverage of the target region. Other types of varying magnetic fields may be induced, such as those having sinusoidal, “sawtooth,” or other profiles over time, any of which profiles may be pre-programmed in the form of instructions to a computer software algorithm.

In alternative embodiment, an initial and temporary cell engraftment phase of an overall wound treatment protocol may be accompanied by the inducement of a relatively high magnetic field, which may result from a combined magnetic field of magnetic particles and the magnetically inducible particles. Inducement can result, for example, in the presence of a surrounding external magnetic field, which may in turn be generated by a varying external electric field. A cell engraftment phase may be followed by a tissue growth phase characterized by a relatively low magnetic field. The use of a combination of magnetic particles and magnetically inducible particles can provide a continuous baseline magnetic field, such that external inducement may not be required over at least a portion (e.g., the majority) of the treatment duration. One type of magnetically inducible particle is an electrically conductive coil that is aligned to induce a magnetic field through the target region, when current flows through the coil.

Accordingly, particular embodiments of the invention are directed to methods for promoting wound repair or reconstruction of damaged tissue in a patient. The methods comprise implanting a plurality of magnetic particles or magnetically inducible particles in the patient proximate a target region, which normally corresponds to the site of a wound (e.g., an articular cartilage defect). The methods also comprise delivering, to the target region, regenerative cells such as mesenchymal stem cells (MSCs) that are complexed with a magnetizable substrate. Following these steps, the complexed cells are within a magnetic field of the magnetic particles, or otherwise the magnetically inducible particles when a magnetic field is induced.

Further embodiments of the invention are directed to kits for promoting wound repair with regenerative cells in a patient. Representative kits comprise (a) magnetizable substrates such as ferromagnetic beads having cell surface recognition ligands (e.g., anti-CD44 antibodies) bonded thereto, (b) magnetic particles such as magnetic spheres having an average diameter of less than about 1 mm and a magnetization of less than about 10,000 A/m, and (c) an elongated implantation device, such as an arthroscope or a needle, that is adapted to implant the magnetic spheres in a tissue of the patient, such as in the subchondral bone directly beneath damaged articular cartilage. These components of the kit may be packaged and/or sold together. In place of, or in addition to, the magnetic particles, the kits may comprise magnetically inducible particles, such as magnetically inducible coils (e.g., electrically conductive coils having an average diameter of less than about 1 cm, or less than about 5 mm). In the case of kits comprising magnetically inducible particles, such kits may also comprise a device (e.g., an article or apparatus) which is adapted to externally induce a magnetic field through the coils or other magnetically inducible particles when implanted in the patient. Such a device may be an article (e.g., a knee brace or wrap) that is further adapted to fit over, or be worn over, a knee or hip joint of the patient.

Further embodiments of the invention are directed to methods for regenerating articular cartilage. Representative methods comprise magnetically isolating or confining MSCs proximate an articular cartilage lesion or other target region of a tissue wound. Magnetic isolation or confinement is performed using a plurality of magnetic particles, such as magnetic spheres. Advantageously, these particles may be implanted in the patient in a three-dimensional profile that is specific to the geometry of the articular cartilage lesion or other wound.

Further embodiments are directed to magnetic particles, such as magnetic spheres, adapted to be implanted in a tissue of a patient, proximate a target region, for example a wound, where cell growth leading to reconstruction of damaged tissue is desired. Representative spheres have an average diameter of less than about 1 mm and may comprise a tissue-compatible substituent such as a bone-compatible polymer. To aid in bonding of the substituent, the surfaces of the magnetic particles may be derivatized with a coupling agent (e.g., a silicon-containing monomer or polymer) that is reactive with the tissue-compatible substituent.

In any of the embodiments described herein, it is understood that all or a portion of the magnetic particles can be substituted with magnetically inducible particles, allowing all or a portion of the total magnetic field from these particles to be varied as discussed above. As discussed with respect to the magnetic particles, the magnetically inducible particles are adapted to be implanted in a tissue of a patient. Therefore, in the case of treating wounds in articular cartilage, the magnetically inducible particles may be adapted to be implanted in the subchondral bone and/or the articular cartilage of patient. Upon implantation, the magnetically inducible particles are oriented such that they generate a magnetic field in the target region (e.g., the wound in the articular cartilage), upon exposure to an externally induced magnetic field (which may in turn be generated by a changing, external electric field). Representative magnetically inducible particles are coils having an average coil diameter (e.g., diameter of their circular cross section) of less than about 5 mm.

Further embodiments of the invention are directed to sheets comprising magnetic materials and having, as with a plurality of magnetic particles, the capability to provide a magnetic field that can be tailored to the geometry of a wound. Accordingly, other embodiments of the invention are directed to methods for promoting wound repair in a patient, where the methods comprise (a) implanting a sheet comprising a magnetic material in the patient, proximate a target region, and (b) delivering, to the target region, regenerative cells complexed with a magnetizable substrate. Following (a) and (b), the regenerative cells complexed with the magnetizable substrate are within a magnetic field of the sheet. In an embodiment where the regenerative cells are placed or adhered onto the sheet ex vivo, and then sheet and cells are implanted together, the above steps (a) and (b) will occur simultaneously. Yet other embodiments of the invention are directed to kits for promoting wound repair, where the kits comprise (a) magnetizable substrates such as ferromagnetic beads having cell surface recognition ligands (e.g., anti-CD44 antibodies) bonded thereto, and (b) a sheet comprising a magnetic material, from which a magnetic implant, conforming to the shape of the wound, may be cut. These components of the kit may be packaged and sold together. Still other embodiments of the invention are directed to methods for regenerating articular cartilage. Representative methods comprise magnetically isolating or confining MSCs proximate an articular cartilage lesion or other target region of a tissue wound, using a sheet of magnetic material that is cut and shaped in a three-dimensional profile that is specific to the geometry of the articular cartilage lesion or other wound.

These and other embodiments and aspects relating to the present invention are apparent from the following Detailed Description.

### BRIEF DESCRIPTION OF THE DRAWINGS

FIG. 1 is a cross-sectional side view of a punctate wound in the articular cartilage of a joint. The wound extends only partially into the thickness of the cartilage but not into the underlying subchondral bone, into which magnetic particles are implanted.

FIG. 2 depicts a cross-sectional side view of a punctate wound in the articular cartilage of a joint, but in this case the wound extends completely through the thickness of the cartilage and even perforates into the underlying subchondral bone, into which magnetic particles are implanted.

FIG. 3 depicts a cross-sectional side view of a blunt wound in the articular cartilage of a joint. As in FIG. 2, the wound extends completely through the thickness of the cartilage and perforates into the underlying subchondral bone. Magnetic particles are implanted into the subchondral bone, as well as in articular cartilage adjacent the boundary of the wound.

FIG. 4 depicts a cross-sectional side view of a wound as depicted in FIG. 1, with a sheet comprising a magnetic material fitting over the wound, and regenerative cells complexed with a magnetizable substrate being present on the surface of the sheet.

FIG. 5 depicts different views of a specific implantation of magnetic particles into a joint surface.

FIG. 6 depicts a cross-sectional side view of the use of magnetic particles attached to “barbs” that anchor them into the subchondral bone.

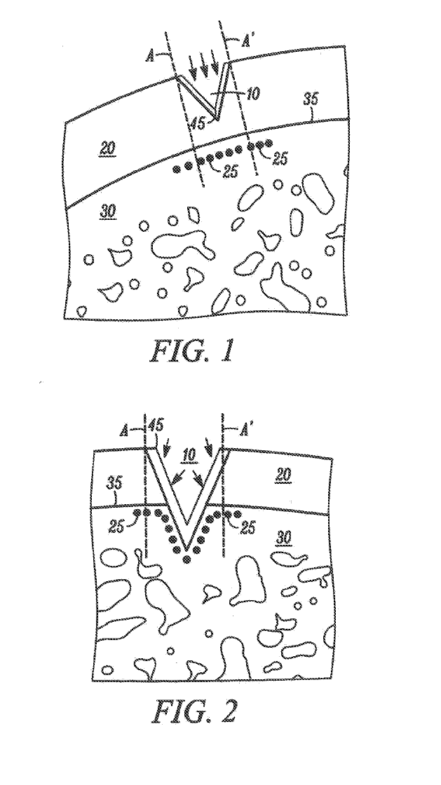

The same reference numbers are used throughout the figures to illustrate the same or similar features. FIGS. 1-4 should be understood to present an illustration of particular aspects of the invention and/or principles involved, without posing any limitation on the invention as defined in the appended claims. In order to facilitate explanation and understanding, simplified illustrations are used, in which the features shown are not necessarily drawn to scale. For example, magnetic particles 25 are shown larger, relative to the dimensions of target region 10, than they might otherwise be according to some embodiments.

### DETAILED DESCRIPTION

### Magnetic Particles and Sheets Comprising Magnetic Material

The magnetic particles that are implanted proximate a target region, such as a wound, may comprise any magnetic material that creates a persistent magnetic field and therefore acts as a permanent magnet at physiological temperatures, i.e., the magnetic material has a Curie temperature T, of greater than about 300° K. Permanent magnets are generally obtained as a result of the magnetic material having undergone special processing in a powerful magnetic field to align its internal microcrystalline structure. Representative magnetic materials include the metals iron, cobalt, nickel, manganese, vanadium, platinum, aluminum, and any of the rare earth metals (lanthanoids), as well as alloys comprising at least one of these metals and compounds comprising at least one of these metals. Alloys include Mn—Al and Pt—Fe alloys, in addition to other types of iron alloys that may be classified as ferrite. Alinco alloys are magnetic materials made by casting or sintering a combination of aluminum, nickel and cobalt with iron and normally small amounts of other elements. Rare earth metals and metal alloys that may be useful as magnetic materials include neodymium, samarium, and their alloys, such as samarium-cobalt and neodymium-iron-boron alloys. Compounds of the above metals include their oxides, such as magnetite (Fe₃O₄) and maghemite (γ-Fe₂O₃), hematite (α-Fe₂O₃), martite (Fe₂O₃), and ferric hydroxide (β-FeOOH) as well as natural magnetic minerals such as lodestone. Other compounds of these metals are carbon-containing compounds, i.e., organometallic compounds, with cyano group-containing compounds being representative. Specific examples are the tetracyanoethylenide forms of these metals (e.g., iron tetracyanoethylenide), optionally solvated.

Alternatively, certain forms of carbon, as well as metal-free organic compounds that exhibit ferromagnetic behavior at sufficiently high temperature, may also be employed as magnetic materials. For example, ferromagnetic pyrolytic carbon containing, in the graphite skeleton, carbon atoms with a significant number of unpaired electrons and/or nitrogen atoms, are exemplary magnetic materials. Five general classes of metal-free, carbon-containing magnetic materials are described in Makarova, T. L., “Magnetism of Carbon-Based Materials,” STUDIES OF HIGH-TC SUPERCONDUCTIVITY, (cond-mat/0207368), 44-45 (2004), namely (i) chains of interacting radicals, (ii) carbonaceous substances with a mixture of sp² and sp³ coordinated atoms, (iii) amorphous carbon structures containing trivalent elements such as P, N, B, (iv) nanographite, nanodiamond, and carbon nanofoam, and (v) fullerenes.

Particular members of any of these classes, with these members being incorporated from this publication herein by reference (e.g., polydiacetylene crystal poly-BIPO, where BIPO is 1,4 bis (2,2,6,6-tetramethyl-4-piperidyl-1-oxyl)-butadiene) can provide the magnetic material used in the magnetic particles. Any of the magnetic polymers (i.e., polymeric magnets) described in this reference may be used as magnetic materials. Other examples of polymeric magnets include magnetic polymeric nanolatexes described, for example, in Deng, Y. et al., JOURNAL OF MAGNETISM AND MAGNETIC MATERIALS 257 (2003): 69-78, hereby incorporated by reference for its disclosure of these polymeric magnets. Further types of polymeric magnets are described in U.S. Pat. Nos. 6,359,051; 5,990,218; 4,881,988; 4,562,019; and 4,327,346, which polymeric magnets are hereby incorporated by reference. A preferred type of polymeric magnetic, for use as a magnetic material, is biodegradable, such as the biodegradable polymeric superparamagnetic nanoparticles described in Chorny, M. et al., THE FASEB JOURNAL, Vol. 21 (August 2007): 2510-2519.

The magnetic particles may further comprise, in addition to any of the above magnetic materials, a base material, thereby resulting in a composite. In such embodiments, the amounts of magnetic and base materials can be varied to achieve desired physical and chemical properties of the composite, as well as a desired magnetization of the magnetic particles. The base material may therefore serve as a diluent of the magnetic material, which may be distributed essentially uniformly throughout the base (e.g., to form a macroscopically homogeneous composite magnetic particle, comprising the base and the dispersed magnetic material). An exemplary ferrite-containing magnetic material composite, for example, is a sintered composite of powdered iron oxide and a barium/strontium carbonate base material. Alternatively, the base material may underlie a coating or deposited layer of the magnetic material, which may, for example, be formed as a thin, uniform layer of a spherical magnetic particle.

Base materials include polymers, for example polystyrene and styrene co-polymers (e.g., styrene-acryl polymers), polyolefins (e.g., polyethylene or polypropylene), polyvinyl chloride, polyurethane, acrylonitrile butadiene styrene, thermoplastic rubbers, ethylene vinyl acetate, and polyesters. Preferred polymers include those that exhibit a capacity for water absorption and are therefore capable of swelling after implantation upon contact with physiological fluids. The size increase accompanying such swelling enhances the ability of these polymers to become secured at the site of implantation, and consequently the stability of the generated magnetic field. Representative swellable polymers are sodium acrylate polymers, acrylamide polymers, acrylamide derivative polymers or copolymers, sodium acrylate-vinyl alcohol copolymers, vinyl acetate-acrylic acid ester copolymers, vinyl acetate-methyl maleate copolymers, isobutylene-maleic anhydride crosslinked copolymers, starch-acrylonitrile graft copolymers, crosslinked sodium polyacrylate polymers, crosslinked polyethylene oxide polymers, and blends thereof. The formation of small polymeric spheres or beads in the nanometer to micron range can be performed according to methods known in the art. When used, the base material of the magnetic particles is often not magnetic or even magnetizable. Other representative base materials include metal oxides.

The magnetic particles may further include a biodegradable substituent (e.g., biodegradable polymer or mineral) and/or a tissue-compatible substituent (e.g., tissue-compatible polymer or mineral). Such substituents can serve as “barbs” or “anchors” that help stabilize the magnetic particles within the desired tissue and prevent their migration, at least temporarily (e.g., over the course of the treatment duration). In the case of a biodegradable substituent, for example, the stabilization is temporary, such that the magnetic particles may be more easily “harvested” or removed from the patient following degradation. Preferably, biodegradable substituents and/or tissue-compatible substituents are attached, for example by covalent bonding, to the surface of the magnetic particles. In some cases this attachment may be facilitated through the use of a coupling agent having points of attachment that are reactive with both the surface of the magnetic particle and the substituent. Coupling agents include silicon-containing monomers, oligomers, and polymers, and especially those having at least two reactive silanol (Si—OH) groups (e.g., polyorganosiloxanes) that provide points of attachment with certain types of materials that are dissimilar in composition (e.g., a biodegradable polymer or mineral and the surface of a magnetic particle). In the absence of a coupling agent, such materials might otherwise be only weakly associated or not at all. When a coupling agent is used, the surface of the magnetic particle may first be derivatized with the coupling agent to provide a source of functional groups, such as silanol groups, carboxyl groups, or amine groups, which are reactive with the tissue-compatible substituent or biodegradable substituent.

Therefore, in the case of magnetic particles that are adapted to be implanted into the subchondral bone of a joint surface, as discussed in greater detail below, these particles may comprise a bone-compatible substituent, such as a bone-compatible polymer or mineral, optionally bonded through a coupling agent. Representative bone-compatible polymers include poly(methyl methacrylate) (PMMA), poly(lactic acid) (PLA), polyglycolide, polycaprolactone, poly(lactic-co-glycolic acid) (PGLA), poly(desamino-tyrosyl-tyrosine ethyl ester), ultra high molecular weight polyethylene (UHMWPE), polyhydroxybutyrate, a polyimminocarbonate, a polyanhydride, a polyethylene glycol/polybutylene terephthalate (PEG/PBT) copolymer, a polyetherurethane, a polyetherketone, a polysulfone, an epoxy resin, a polycarbonate, and a poly(ortho ester). Representative bone-compatible minerals include hydroxyapatite, apatite-wollastonite glass ceramic, crystalline and amorphous calcium phosphates, and mixed metal oxides (e.g., the mixture of SiO₂, Na₂O, CaO, and P₂O₅ known as Bioglass).

The individual magnetic particles are generally small in size, relative the size of the target region through which a magnetic field is directed, according to the treatment methods described herein. The total area over which the particles are implanted, however, may be comparable to the area of the target region or even exceed this area. In one embodiment, the magnetic particles are substantially spherical and have an average diameter that is less than 50%, less than 25%, less than 10%, or less than 5%, of a maximum dimension of the target region, such as the wound. The maximum dimension of the target region may be, for example, the longest line segment adjoining the boundaries of a wound or lesion. In alternative embodiments in which the magnetic particles have other shapes, such as flat disk shapes, the largest dimension of these particles (e.g., corresponding substantially to the diameter of a circular cross section, or the length of a major axis of an elliptical cross section, of the disk) is less than 50%, less than 25%, less than 10%, or less than 5%, of a maximum dimension of the target region. In terms of absolute size, the average diameter of spherical magnetic particles, or otherwise the average largest dimension of magnetic particles having other shapes (e.g., in the case of a cylindrical shape, the diameter of a cylindrical cross section or the length of a cylinder, whichever is longer), may be from about 1 micron (μm) to about 2 mm, from about 1 μm to about 1 mm, or from about 1 μm to about 500 μm. In other embodiments, the magnetic particles may be nanosized, having an average diameter or average largest dimension from about 10 nanometers (nm) to about 1000 nm.

Apart from being relatively small in size, the magnetic particles are generally implanted in the immediate vicinity of the target region, for example at an average distance that is less than about 10 mm, less than about 5 mm, less than about 3 mm, or less than about 1 mm, from the target region. In other embodiments, all of the magnetic particles, at least about 90%, or at least about 80%, of the magnetic particles, are implanted within these distances from the target region. In specific embodiments in which the target region is an articular cartilage wound, and particularly in a knee or hip joint, all, or any of these portions, of the magnetic particles may be implanted within these distances below or beneath the target region, or otherwise within these distances below or beneath a bone surface of the knee or hip joint (e.g., below the tibial surface).

In this disclosure, the significance of the positional modifiers “below” or “beneath” refers to elevations in the case of the patient standing in an upright position. However, regardless of their positions (e.g., above, below, or to the side) relative to the wound, the magnetic particles (or magnetically inducible particles as discussed below) may be implanted close to, and in a manner that conforms substantially to, the shape of the target region. In this manner, the resulting, three-dimensional profile of the implanted particle cluster can provide a magnetic field having a strength and direction that is tailored to the wound geometry, whereby the attractive forces effectively retain regenerative cells in the target region. The greater the number of magnetic particles, the greater the degree to which the resulting magnetic field can be matched to the features of the target region, thereby avoiding large fluctuations in magnetic field strength that can hinder the desired, uniform placement and growth of regenerative cells over the surface of this region. The magnetic particles may be implanted, for example, at a coverage ratio from about 10 to about 10,000, or from about 10 to about 1000, particles per square centimeter (cm²) of area of the target region. Due to their number and proximity to the target region, the magnetization (or magnetic moment per unit volume), of the magnetic particles is generally small in comparison to conventional, internal or external magnets used in medical applications. In particular embodiments, the magnetic particles have an average magnetization from about 1 to about 10,000 amperes/meter (A/m), from about 1 to about 5,000 A/m, or from about 10 to about 1,000 A/m.

In yet further embodiments, any of the above base materials may serve as a matrix material for binding some or all of the magnetic particles, for example in the form of a sheet of matrix material having the discreet magnetic particles contained therein. Such a sheet of matrix material is not macroscopically homogeneous, due to the change in composition that occurs where the magnetic particles are located. The change in composition is also accompanied by a localized change in magnetic field. Alternatively, the magnetic material may be dispersed uniformly throughout a thin sheet of matrix material (e.g., a flexible elastomer or a biodegradable polymer), such that discreet particles are no longer perceptible and the sheet of matrix material and magnetic material is macroscopically homogeneous. Such a uniform distribution of magnetic material can provide a correspondingly uniform magnetic field over the surface of the sheet. A similar result can be achieved using a layer of the magnetic material (e.g., a layer of magnetic material powder) on the surface of the matrix or within the matrix, whereby a concentration gradient of the magnetic material is present across the thickness of the sheet, but not in any direction of the planar area of the sheet. In this case, the magnetic field over the surface of the sheet is uniform as well. Finally, a magnetic material in the form of a sheet can also be obtained without the use of a matrix material in some cases. For example, sheets of magnetic metal or alloy materials, or otherwise sheets of organometallic magnetic materials or metal-free, carbon-containing magnetic materials (e.g., polymeric magnets), as described above may also be used. If a matrix material is used, it should generally impart both strength and flexibility properties to the resulting sheet (e.g., at representative average sheet thicknesses given below), such that it can be formed (e.g., cut, stamped, bent, and/or folded) into a desired shape that conforms to a three-dimensional profile of the target region. If no matrix material is used, then such properties are preferably obtained from the magnetic material itself, at representative sheet thicknesses given below. Polymeric magnets, and especially biodegradable polymeric magnets as described above, are preferred magnetic materials for forming a sheet, if no matrix material is used.

By “sheet” is meant a structure having at least one, and preferably only one, dimension that is much smaller than its other dimensions. The average thickness of a representative sheet is less than about 3 mm (e.g., from about 100 nm to about 3 mm), less than about 1 mm (e.g., from about 1 μm to about 1 mm), or less than about 500 μm (e.g., from about 50 μm to about 500 μm). A sheet comprising a magnetic material as described above, and optionally a matrix material as described above, can be used to attain the same objective, as discussed above with respect to the magnetic particles or magnetically inducible particles, of conforming a magnetic field to the three-dimensional structure of a wound or other target region where cell engraftment and growth are desired. The use of a sheet comprising a magnetic material may be advantageous in embodiments where a sheet can be cut, as well as shaped and/or stamped in three dimensions, to match the contours of the wound, where cells are to be magnetically confined. The characteristics of a sheet which allow for its ability to be cut and shaped, as required in the treatment methods described herein, include the flexibility of the matrix material(s) and/or magnetic material(s) as discussed above, in addition to the thickness dimension being small, relative to its length and/or width dimensions. For example, the ratio of the surface area, in cm², of a sheet comprising a magnetic material, for implantation proximate a target region according to the methods described herein, to its thickness, in cm, will generally exceed 10, but will typically exceed 100, and will often exceed 1,000 or even exceed 10,000. Flexible magnets in the form of a sheet are available, for example, from Dura Magnetics, Inc. (Sylvania, Ohio USA).

In specific embodiments, the matrix material, or even the entire sheet, may be biodegradable, such that over an extended time (e.g., after engraftment) there are no foreign bodies remaining in the target region. As with the use of magnetic particles, the magnetic field generated by the sheet is uniform and relatively weak, having a magnetization as discussed above with respect to the magnetic particles. This minimizes detrimental effects on the cells that can occur in the presence of higher magnetization, as discussed above, and prevents cell agglomeration or clumping.

Depending on the location and type of wound, it may be advantageous to implant more than one sheet, with each sheet being proximate different sections of the target region. It is also possible to combine the implantation of one or more sheets as described above with either or both of the magnetic particles or magnetically inducible particles, as described herein.

### Magnetically Inducible Particles

The ranges discussed above for magnetic particles, with respect to their proximity of implantation to the target region and their magnetization, also apply to magnetically inducible particles. In the case of the magnetization ranges given above, these apply to the magnetically inducible particles when a magnetic field is induced, and otherwise these particles generally exhibit no magnetization. The coverage ratios discussed above likewise apply to the magnetically inducible particles, where the number of particles per cm² of the target region area would in this case refer to the total number of both magnetic and magnetically inducible particles.

In addition, the sizes, in relation to the size of the target region as well as in absolute terms, discussed above with respect to magnetic particles, also apply to magnetically inducible particles. However, in some cases the fabrication issues relating to magnetically inducible coils and the need to orient the coils properly at a wound site may warrant larger sizes. In some embodiments, therefore, if the magnetically inducible particles are in the form of coils, then at least some of these coils (e.g., at least 5%, at least 10%, or at least 20%), or possibly all of the coils may have a coil diameter of greater than about 5 mm (e.g., from about 5 mm to about 3 centimeters (cm) or from about 5 mm to about 2 cm). According to other embodiments, a relatively large proportion (e.g., at least 50%, at least 80%, or at least 90%) of the implanted coils may have a coil diameter of less than about 5 mm (e.g., from about 500 ∥m to about 5 mm or from about 1 mm to about 5 mm). Electrically conductive coils (e.g., microcoils) are a preferred form of the magnetically inducible particles, which may also be referred to as electromagnetic wire coils that act as magnets when an electric current is passed through them. The largest dimension of such coils may be the diameter of their circular cross sections or the length of their axes. When implanted, the coils are aligned to induce a magnetic field through the target region, if an electric current is established in these coils. Accordingly, a given coil is generally oriented such that an imaginary line perpendicular to its circular cross section, and corresponding to a magnetic field line induced by the coil, is substantially perpendicular to the surface of the target region that is closest to that coil.

The use of magnetically inducible particles allows the magnetic field in the target region to be varied as desired, by establishing a varying electric current in the coils. For example, a changing external electrical field (causing an external magnetic field) may be applied from outside the target region, and preferably from outside of the body such as from a knee wrap or other article that may be worn or placed over the site of the wound or other region where cell engraftment and growth is desired. Otherwise, an external apparatus need not surround the target region if its location (e.g., within the hip joint) renders this difficult, as long as the apparatus is capable of generating a changing electric field (causing an external magnetic field) that in turn induces a magnetic field through the target region, as dictated by the placement of the implanted, magnetically inducible particles. Variations in the external magnetic field, provided by such articles or apparatuses, lead to variations in the current through the magnetically inducible coils and consequently the magnetic field through the target region. Representative variations in magnetization of the magnetically inducible particles correspond to the ranges in magnetization as discussed above with respect to magnetic particles.

By implanting a combination of magnetic particles and magnetically inducible particles, a constant, baseline magnetic field is established in the target region, which may be varied (e.g., augmented or reduced, or even completely offset) according to the design of a specific treatment protocol. For example, a magnetic field may be induced in magnetically inducible coils in a temporary, beginning phase to initiate engraftment of regenerative cells such as stem cells. In this phase, the overall magnetic field in the target region is augmented, relative to the baseline magnetic field, corresponding to that generated by the magnetic particles alone. The beginning phase may then be followed by a longer tissue regeneration phase, in which the baseline magnetic field is sufficient to maintain the engrafted cells in the target region, without the need for using an external article or apparatus as discussed above.

### Magnetizable Substrate-Complexed, Regenerative Cells and Growth Factors

Regenerative cells refer to any cells that are capable of growing tissue that is native to the site of the target region or wound. Such cells therefore include those corresponding to the damaged tissue itself, such as chondrocytes in the case of repairing a wound to damaged hyaline cartilage of the target region. Other cells are those that are at least partially differentiated into cells corresponding to those of the damaged tissue. Specific cell types that can be used to populate target regions therefore include neurons for the growth of tissues of the brain, spinal cord, retina, and acoustic and peripheral nerves; vascular endothelial cells for growth of tissues of blood vessels and the heart; pancreatic islet cells for growth of tissues of the pancreas, and other cell types that will be appreciated by those skilled in the art, having knowledge of the present disclosure.

Regenerative cells also include stem cells, such as mesenchymal stem cells (MSCs), embryonic stem cells, and induced pluripotent stem cells. MSCs can be separated from a tissue sample of the patient, such as a blood or bone marrow sample, prior to their use in the treatment methods described herein, including wound repair. Synovium-derived mesenchymal stem cells (SDMSCs), mentioned above, represent a particular type of regenerative cells. MSCs and other types of stem cells may be grown in their undifferentiated state ex vivo, through mitotic expansion in a specific growth medium. Growth may be conducted in adherent or suspension culture, although a free flowing or suspension culture may be desirable in terms of the ability to harvest cells in a less differentiated state. In the case of MSCs, the harvested cells can then be activated to differentiate into bone, cartilage, and other connective tissues (e.g., tendons and ligaments), depending on their environment, including the presence growth factors as well as other types stimuli that may be biochemical, cellular, or mechanical in nature. In cases of repairing wounds to other tissues, stem cells other than MSCs that are committed to growing the appropriate tissue types may be used.

When the regenerative cells are delivered to the target region, they are in the form of a complex with a magnetizable substrate. This substrate is of a type often referred to in the art as a “magnetic bead,” although in general the substrate is only magnetic in the presence of a magnetic field and (unlike the magnetic particles, magnetically inducible particles, or sheets discussed above) does not generate a magnetic field to any appreciable extent. The magnetizable substrate may comprise any of the magnetic materials as discussed above with respect to the magnetic particles, with the only difference being that these magnetic materials are preferably not permanently magnetized, and are therefore referred to here as “magnetizable materials.” In particular embodiments, the magnetizable substrate comprises a ferromagnetic material, a ferrimagnetic material, or a paramagnetic material, which magnetizable material may be a metal, a metal alloy, or a metal compound such as a metal oxide, metal carbonate, or metal silicate. In yet other embodiments, the magnetizable substrate comprises a diamagnetic material (e.g., pyrolytic carbon or pyrolytic graphite) that opposes an applied external magnetic field. In addition, the magnetizable substrate may further comprise a base material as discussed above with respect to the magnetic particles, such that the magnetizable material may be distributed uniformly throughout such base material (e.g., to form a macroscopically homogeneous composite magnetizable substrate, comprising the base and the dispersed magnetizable material), or may otherwise be coated on the underlying base material, for example in the form of a sphere. Representative base materials include polymers such as polystyrene and styrene copolymers (e.g., styrene-acryl copolymers), as well as biodegradable polymers including poly(lactic acid) and copoly(lactic acid/glycolic acid). Other polymers useful as base materials for the magnetizable substrate include those described above as possible base materials for the magnetic materials.

The magnetizable substrate may be fabricated in spherical form with a wide range of diameters, with representative average diameters ranging from about 10 μm to about 1000 μm, from about 30 μm to about 800 μm, and from about 40 μm to about 500 μm. Such sizes are advantageous in that they enable the magnetizable substrate and their complexed stem cells and/or growth factors (discussed below) to be easily injectable through arthroscopic devices or other elongated implantation devices, having injection diameters of 5 mm or less. The use of an arthroscope, having the ability to illuminate and provide a visual image of the target region, allows for accurate and effective delivery of the complexed regenerative cells and together with any required, complexed growth factor, into the target region. It is also possible to use nano-sized spheres of the magnetizable substrate, having average diameters, for example, in the range from about 10 nm to about 1000 nm.

Representative magnetizable substrates therefore include ferumoxides, often in the form of ferumoxide-protamine sulfate (FePro) beads, which are approved by the U.S. Food and Drug Administration for use in humans. Other commercial products are available, such as those used in Magnetic Resonance Imaging (MRI), for example silica-coated super paramagnetic iron oxide nanoparticles (SPION) contrast agents. Types of magnetizable substrates include polymer (e.g., polystyrene) particles of various sizes, which are coated with a ferromagnetic material, a ferromagnetic material, or a paramagnetic material, such as the spherical particles available under the trade name Dynabeads® (Invitrogen Life Technologies, Grand Island, N.Y., USA). Other magnetizable substrates include beads of a polymer or copolymer base material, or otherwise a metal oxide (e.g., silica) base material, which is coated or impregnated with a metal selected from the group consisting of iron, cobalt, nickel, manganese, vanadium, platinum, aluminum, a rare earth metal (lanthanoid), or an alloy comprising at least one of these metals. Further representative magnetizable substrates are therefore those available under the trade name PureProteome™ (EMD Millipore, Billerica, Mass., USA). Other specific examples of magnetizable substrates include those comprising styrene-acryl copolymer base materials that are coated with a thin film of ferrite, such as those available from Ferrisphere, Inc. (Flanders, N.J., USA).

The formation of a complex between the regenerative cells and the magnetizable substrate can occur via interactions between groups on their respective surfaces. Possible interactions include antibody-antigen binding, peptide adhesion, covalent bonding, electronic (e.g., ionic) association, hydrogen bonding (e.g., hybridization), or a combination of such interactions. In order to obtain a satisfactory degree and specificity of complexing with a desired regenerative cell type, it is generally necessary to attach a cell surface recognition ligand to the surface of the magnetizable substrate. Such a cell surface recognition ligand is preferably specific to a cell surface marker of the desired regenerative cells, or a desired phenotype of these cells. In the case of using MSCs as the regenerative cells, for example, a preferred cell surface recognition ligand is an anti-CD44 antibody or an anti-Human Leukocyte Antigen (HLA) antibody that is specific for the binding of the surface CD44 glycoprotein or HLA of the MSCs. In other embodiments, the cell surface recognition ligand may promote more generalized binding, such as in the case of a cell surface adhesion peptide. Regardless of the ligand used or its specificity for the desired, regenerative cells, it is often necessary to derivatize the surface of the magnetizable substrate to aid in the attachment of this ligand. For example, this surface may be derivatized with carboxyl groups or amine groups that are reactive with any amine groups or carboxyl groups, respectively, of the ligand. The amine-carboxyl reaction forms strong, covalent amide bonds between the ligand and magnetizable substrate. Many other types of surface derivatization and bonding are possible, depending on the specific ligand and magnetizable substrate for a given application.

In embodiments in which the ligands are sufficiently specific to the desired regenerative cells, the magnetizable substrate may advantageously be used to initially isolate these cells from a patient tissue sample. In this manner, the regenerative cell-magnetizable substrate complex can be prepared in conjunction with the necessary cell purification. In representative embodiments of the invention, therefore, prior to the delivery of this complex to the target region and generally also prior to the implantation of the magnetic particles or magnetically inducible particles, the complex is prepared from a tissue sample (e.g., a blood or bone marrow sample) of the patient that contains the regenerative cells. If the appropriate cell surface recognition ligand is attached to the magnetizable substrate, the preparation of this complex can include selectively complexing the regenerative cells (e.g., stem cells of a desired phenotype) through an interaction between this ligand and a surface marker of the phenotype. In the case of MSCs being the desired stem cell phenotype, for example, the preferred interaction is an antibody-antigen binding interaction that is specific to the surface CD44 glycoprotein or the surface HLA of MSCs, as discussed above.

In a similar manner, a magnetizable substrate may also be attached to any of a number of desired growth factors, rather than ligands. This attachment may likewise occur through the derivatization of the surface of the magnetizable substrate. Again, the incorporation of carboxyl, amine, or other functional groups on this surface can be performed to allow covalent (e.g., amide) bond formation with reactive groups of the desired growth factor(s). In particular embodiments, therefore, following (a) implantation of the magnetic particles or magnetically inducible particles and (b) delivery of the regenerative cells, complexed with the magnetizable substrate, to the target site, one or more growth factors that is/are also complexed to a magnetizable substrate, may be in the target region and within the same magnetic field acting on the regenerative cell-magnetizable substrate complex. In this manner, the regenerative cells and the appropriate growth factors for stimulation of tissue regeneration and wound healing can be magnetically confined together in the target region.

In the case of a magnetizable substrate that is a diamagnetic material such as pyrolytic graphite, the regenerative cells and optionally their growth factors could be magnetically repelled from (e.g., levitated away from) implanted magnetic particles or magnetically inducible particles. This may be advantageous when the target region is more easily accessed by regenerative cells from below, and it is desired to implant the magnetic particles or magnetically inducible particles in an opposing surface directly below the target region. Therefore, for example, such particles could be implanted in the knee meniscae or tibial plateau to magnetically force regenerative cells, complexed to diamagnetic materials, toward a target region on the underside of the distal femoral condyle. In fact, according to some embodiments in which regenerative cells are complexed with diamagnetic materials, implanted magnetic particles or magnetically inducible particles might not be necessary, as an externally applied (e.g., surrounding) magnetic field alone might be sufficient to confine such complexed cells by magnetic repulsion. Alternative embodiments of the invention may therefore constitute any of the methods described herein, but comprising, rather than a step of implanting a plurality of magnetic particles or magnetically inducible particles in the patient, proximate the target region, a step of establishing a magnetic field external to a target region (e.g., external to the patient) sufficient to magnetically confine the regenerative cells, complexed with the magnetizable substrate, within the magnetic field.

The growth factor(s), when used, may be complexed to the same or a different type of magnetizable substrate that is used to complex the regenerative cells. In some embodiments, it may be possible to derivatize the surface of a magnetizable substrate with one or more types of functional groups (e.g., carboxyl groups and/or amine groups) and then attach cell surface recognition ligands to only a portion of the available groups or bonding sites. The remaining, free bonding sites would then be available for attachment with one or more desired growth factors, such that growth factors and regenerative cells would be complexed to the same substrate particle, resulting in close interactions. Therefore, to achieve a desired ratio of regenerative cells:molecules of growth factor (e.g., from 1000:1 to 1:1000, from 100:1 to 1:100, or from 10:1 to 1:10), it may be possible to vary the ratio of ligand-attached functional groups to free functional groups on the one or more types of magnetizable substrates used to complex the regenerative cells and the growth factors. In some cases, this ratio may be manipulated by using differing amounts of one type of substrate having substantially all ligand-attached functional groups and another type of substrate having substantially all free functional groups. The functional groups used to attach ligands and growth factors may be the same or different.

Representative growth factors, which can stimulate tissue regeneration from regenerative cells, including the differentiation of stem cells into cells of the desired tissue, include cytokines, transforming growth factors (TGF-α, TGF-β), bone morphogenetic protein (BMP), fibroblast growth factor (FGF), insulin-like growth factor (IGF-I), granulocyte-macrophage colony stimulating factor (GM-CSF), macrophage colony stimulating factors (M-CSF, CSF-1), granulocyte colony stimulating factor (G-CSF), erythropoietin, thrombopoietin, hematopoietic stem cell factor (SCF), monocyte chemotactic activity factor (MCAF), epithelial growth factor (EGF), platelet-derived growth factor (PDGF), and nerve growth factor (NGF). Specific growth factors that are chondroinductive agents include glucocorticoids such as dexamethasone; members of the TGF-β superfamily such as a bone morphogenic proteins (e.g., BMP-2 or BMP-4), TGF-β1, inhibin A or chondrogenic stimulating activity factor; components of the collagenous extracellular matrix such as collagen I (e.g., in gel form); and a vitamin A analog such as retinoic acid.

### Implantation of the Magnetic Particles and Delivery of Regenerative Cells

The regenerative cells that are complexed with a magnetizable substrate, as described above, are delivered to the target region (e.g., wound), prior to, after, or simultaneously with, implantation of the magnetic particles or magnetically inducible particles, or otherwise a sheet comprising a magnetic material, as described above. Preferably, the cells are delivered to the target region following the implantation, which, in the case of magnetic particles or a sheet, establishes the desired magnetic field surrounding the target region. The subsequent delivery of the complexed, regenerative cells, in this case, ensures that they are attracted from the outset to the desired areas, thereby eliminating any transient period in which the delivered cells can migrate essentially freely. In any event, following the implantation and delivery, the regenerative cells (and optionally one or more growth factors described above) complexed with the magnetizable substrate are within the magnetic field of the magnetic particles, the sheet, or of the magnetically inducible particles when the magnetic field is induced. The influence of the magnetic field, whether established before, after, or at the same time as, the delivery of the complexed, regenerative cells, effectively confines these cells to the target region, at least to a greater extent than in the absence of the magnetic field. That is, with all other variables being constant, the population of cells initially delivered to the target region would be greater over time in the presence of the magnetic field, compared to the same population in the absence of the magnetic field. The ability of the magnetic field to maintain/isolate the delivered cells in the target region effectively promotes their multiplication in this region and consequently the regrowth and repair of damaged tissue.

Conveniently, in some embodiments, the sizes of the magnetic particles and/or magnetically inducible particles, in addition to the size of the magnetizable substrates, allows particle implantation and cell delivery to be accomplished with minimal invasion, such as by the use of a needle, catheter, arthroscope, or other elongated implantation or injection device appropriate for the sizes of the particles and magnetizable substrates. The magnetic particles, for example, may be injected through an elongated implantation device such as a syringe/needle assembly that is adapted for their implantation into subchondral bone and/or into hyaline cartilage at varying depths surrounding the target region in a patient. Representative devices for particle implantation and complexed cell delivery therefore include needles, catheters, and arthroscopes having an injection diameter corresponding to a gauge value from about 10 to about 30 gauge (i.e., from about 2.7 mm to about 0.16 mm inner diameter), and preferably from 18 to about 25 gauge (i.e., from about 0.84 to about 0.26 mm inner diameter). In other embodiments, the elongated implantation device is an arthroscopic device that can project an image of the target region and irrigate it. This instrument may be adapted to not only implant the magnetic spheres or magnetically inducible particles in a tissue (e.g., subchondral bone and/or hyaline cartilage) of the patient, but also to deliver the regenerative cells, complexed with the magnetizable substrate, optionally together with one or more growth factors, as discussed above. The injection diameter of an arthroscopic device is typically 5 mm or less. Regardless of the specific type of elongated implantation device or cell delivery device, the particles, cells, and growth factors may be implanted or injected together with appropriate physiological solutions to aid in their transfer to the patient.

The target region, to which the complexed, regenerative cells are delivered and about which the magnetic particles, sheet, or magnetically inducible particles are implanted, can be generally any wound or lesion in a tissue of a patient that is to be treated according to the methods described herein. Representative tissues in which the target region may be located include bone tissues, (e.g., tissues of the femur, tibia, or a rib), articular cartilage, periosteum, heart tissues, spinal cord tissues, pancreatic tissues, kidney tissues, liver tissues, eye tissues, brain tissues, bladder tissues, (e.g., tissues of the gall bladder), prostate tissues, breast tissues, intestinal tissues, skeletal muscle tissues, and lung tissues, as well as connective tissues such as those of tendons and ligaments.

### EXAMPLES

According to specific examples, the target region is a wound in articular cartilage of a joint of the patient, such as a knee, hip, or shoulder joint. The articular cartilage may be thinned or denuded in the target region, or have some other defect. Therefore, in particular embodiments in which articular cartilage repair is the objective, all or at least a portion of the magnetic particles, sheet, and/or magnetically inducible particles may be implanted in subchondral bone of a joint surface that is normally covered by articular cartilage in the case of a healthy joint. Particular examples include knee joint surfaces, such as the surface of a femoral condyle (e.g., the lateral or medial femoral condyle). Other knee joint surfaces include those of the lateral or medial tibial plateau, such as the head of the tibia or fibula. Representative hip joint surfaces include those of the femoral head or the acetabulum. Those skilled in the art will appreciate, in view of this disclosure, the applicability of the methods described herein to other joint surfaces, including those of shoulder and wrist joints. Particular embodiments of the invention therefore relate to methods for regenerating articular cartilage in a patient, and especially hyaline cartilage that is not regenerated in conventional surgical methods for joint repair. These methods comprise magnetically isolating MSCs proximate an articular cartilage wound or lesion, using a plurality of magnetic particles (e.g., spheres) and/or magnetically inducible particles (e.g., coils), and/or a sheet comprising a magnetic material, as described above. The particles may be implanted in tissues surrounding the wound or lesion in a three-dimensional profile that is specific to the geometry of the wound or lesion. For example, it is also possible for all or at least a portion of the magnetic particles and/or magnetically inducible particles and/or a sheet to be implanted in the articular cartilage itself, such as in healthy cartilage immediately adjacent the target region or wound.

In the case of wound repair in the joints, the methods described herein are applicable to the treatment and restoration of any joint tissues, in addition to those described above, that may become damaged. In the case of the knee joint, for example, representative joint tissues include the lateral and medial menisci, the anterior and posterior cruciate ligaments, the lateral and medial collateral ligaments, the patella and patellar tendon, and other tissues of the knee joint which may become wounded or damaged. Likewise, tissues of the hip joint, including hip tendons, ligaments, and bursae, may be similarly treated.

FIGS. 1-3 illustrate specific embodiments of the invention, for the treatment of target regions 10 of damaged or missing articular cartilage. In FIG. 1, target region 10 is a punctate wound in articular cartilage 20 covering the surface of subchondral bone 30. A plurality of magnetic particles 25 is implanted in subchondral bone 30 proximate target region 10, substantially in alignment with the bone surface 35. Magnetic particles 25 in this embodiment extend somewhat beyond lateral boundaries A-A′ of target region 10. Regenerative cells complexed with a magnetizable substrate 45, such as complexed MSCs, are delivered to, and form a surface layer in, target region 10. Due to the magnetic attraction from implanted magnetic particles 25, complexed regenerative cells 45 are attracted substantially in a downward direction onto the wound surface, as indicated by the arrows in FIG. 1.

In the embodiment of FIG. 2, punctate wound of target region 10 extends deeper, compared to the wound illustrated in FIG. 1, and perforates into subchondral bone 30. Again, complexed regenerative cells 45 form a surface layer in target region 10. Magnetic particles 25 near lateral boundaries A-A′ of target region 10 are implanted substantially in alignment with bone surface 35, extending somewhat beyond the lateral boundaries. In addition, in a central section of target region 10, where subchondral bone 30 is affected, magnetic particles 25 are implanted substantially in alignment with the geometry of the wound to subchondral bone 30. In this case, therefore, due to the magnetic attraction from implanted magnetic particles 25, complexed regenerative cells 45 are attracted substantially in a downward direction onto the wound surface, as indicated by the arrows in FIG. 2. Where magnetic particles 25 conform more closely to the wound geometry, it can be appreciated from FIG. 2 that the magnetic force acts substantially normal to the wound surface, which advantageously hinders “puddling” or agglomeration of the delivered, complexed regenerative cells in the central section of the wound or in any other depressed section. This provides an improved distribution of complexed regenerative cells 45 and promotes uniform tissue regeneration throughout target region 10.

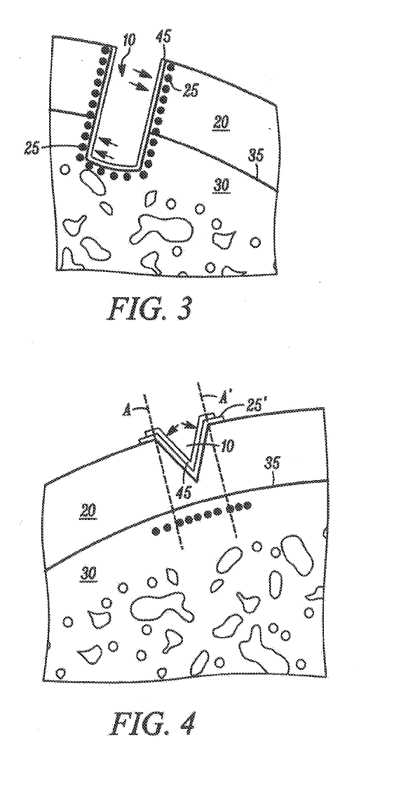

In the embodiment of FIG. 3, all of magnetic particles 25 are implanted such that they conform closely to the geometry of the wound, which in this example is a blunt wound that perforates into subchondral bone 30. Therefore, a portion of magnetic particles 25 is implanted in articular cartilage 20 and another portion is implanted in subchondral bone 30. An advantage of this configuration of magnetic particles 25 is that the magnetic forces acting on complexed regenerative cells 45 are substantially normal to the wound surface, throughout target region 10, encouraging the desired monolayer cell growth. FIGS. 1-3 therefore provide specific examples to illustrate how the use of a plurality of magnetic particles provides significant flexibility in terms of establishing an overall magnetic field in target region 10 for the magnetic confinement or isolation of complexed regenerative cells, thereby preventing cell migration from target region 10 and promoting cell growth and tissue regeneration in this region. All or some of the magnetic particles 25 in the embodiments of FIGS. 1-3 can alternatively by replaced with magnetically inducible particles, or a sheet comprising magnetic material, as discussed above, to achieve the same effects. The use of magnetically inducible particles adds the ability to vary the magnetic field over time.

In the alternative embodiment of FIG. 4, a sheet comprising a magnetic material 25′ replaces magnetic particles 25 in FIGS. 1-3. The flexibility of sheet 25′ allows it to be pressed into a shape conforming to contours of target region 10. Otherwise, sheet 25′ may be pre-formed into the desired shape, such that it may be more gently placed into target region 10, without requiring significant pressure to obtain the desired fit. The sheet may be pre-formed with the aid of an image of target region 10, and possibly a computerized forming apparatus (e.g., a three-dimensional printer) using data from an image as an input. In the embodiment of FIG. 4, sheet 25′ is disposed directly above a wound surface, and complexed regenerative cells 45 are delivered onto sheet 25′ to which they are magnetically attracted. It is also possible for complexed regenerative cells 45 to be delivered ex vivo directly onto sheet 25′ and then the sheet 25 and associated cells 45 implanted in target region together. According to such methods, therefore, the implantation and delivery steps occur simultaneously. An adhesive such as fibrin may be used to firmly implant sheet 25′ in the desired location, and optionally an appropriate coupling agent can be used to bond the fibrin adhesive to sheet 25′. The fibrin may over time be resorbed. Moreover, in a preferred embodiment, sheet 25′ itself comprises a resorbable or biodegradable material, such as a biodegradable polymeric magnet as described above.

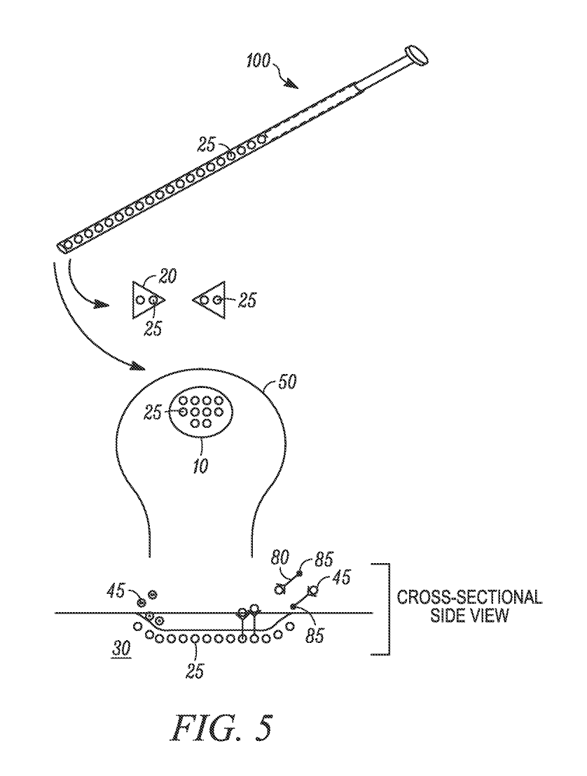

FIG. 5 depicts arthroscopic device 100 capable of implanting magnetic particles 25 (e.g., magnetic beads having a diameter of <1 mm) or magnetically inducible particles. Preferably, arthroscopic device 100 has fiber optic guidance capability, as well as the ability to irrigate/flush target region 10 and surrounding regions of the joint space. In this illustration, target region 10 is a hyaline cartilage defect in the femoral head 50, and magnetic particles 25 are placed into subchondral bone 30 below target region 10. As further shown, magnetic particles 25 are additionally implanted into articular cartilage 20, which in this illustration includes cartilage of the meniscus surrounding femoral head 50. The cross-sectional side view of FIG. 5 further illustrates individual regenerative cells 45, which are magnetically attracted to magnetic particles and therefore stabilized in target region 10. Regenerative cells 45 on the right-hand side of this view are complexed, via binding that is mediated by antibodies 80, to a magnetizable substrate, namely ferromagnetic beads 85. Regenerative cells 45 on the left-hand side of this view have ferromagnetic beads 85 that are internalized within these cells 45.

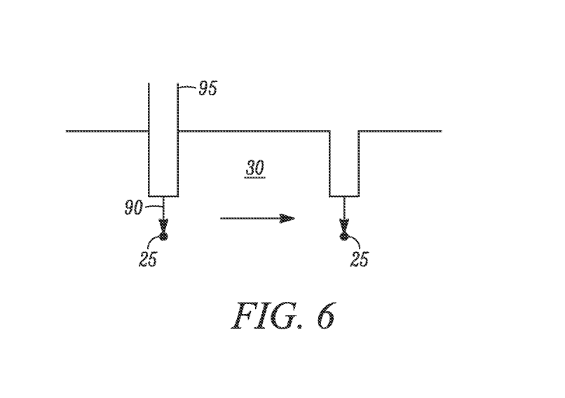

FIG. 6 illustrates the use of magnetic particles 25 attached to “barbs 90,” which, as discussed above, anchor magnetic particles 25 into the subchondral bone 30. Such barbs may be driven below the surface of subchondral bone using a needle-like plunger 95. Implantation of magnetic particles 25 and attached barbs 90 below the surface of subchondral bone 30 can advantageously decrease the amount of wear on a contralateral surface.

Overall, aspects of the invention are directed to the use of magnetic particles, magnetically inducible particles, and/or a sheet comprising magnetic material, to tailor a magnetic field as needed to effectively retain magnetized cells in a region where cell growth is desired, such as an internal tissue wound or lesion. The extent to which the magnetic field may be customized can be improved by imaging the site of a wound or lesion to ascertain its three-dimensional shape. In addition, data obtained from the imaging can be input into an appropriate computer software algorithm that determines the proper placement and strengths of magnetic particles, magnetically inducible particles, and/or sheet(s) comprising a magnetic material to provide an effective magnetic field. Other inputs to such a computer algorithm could include the type of regenerative cells, growth factors, and magentizable substrates used, in addition to other factors. Those having skill in the art, with the knowledge gained from the present disclosure, will recognize that various changes could be made in the methods, kits, magnetic and magnetically inducible particles, and sheets comprising magnetic materials, as described herein, without departing from the scope of the present invention.

1. A method for promoting wound repair in a patient, the method comprising:
(a) implanting a plurality of magnetic particles or magnetically inducible particles in the patient, proximate a target region; and
(b) delivering, to the target region, regenerative cells complexed with a magnetizable substrate,
wherein, following steps (a) and (b), the regenerative cells complexed with the magnetizable substrate are within a magnetic field of the magnetic particles, or of the magnetically inducible particles when the magnetic field is induced.

2. The method of claim 1, wherein:
(i) the regenerative cells are stem cells or chondrocytes;
(ii) following steps (a) and (b), the regenerative cells complexed with the magnetizable substrate are within a magnetic field, together with one or more growth factors complexed with the magnetizable substrate;
(iii) following steps (a) and (b), the regenerative cells complexed with the magnetizable substrate are within a magnetic field, together with one or more growth factors complexed with the magnetizable substrate, wherein the one or more growth factors are selected from the group consisting of cytokines, transforming growth factor-beta (TGF-62 ), bone morphogenetic protein (BMP), fibroblast growth factor (FGF), insulin-like growth factor (IGF-I), and combinations thereof;
(iv) the target region is a wound or lesion in a tissue of the patient selected from the group consisting of bone tissue, articular cartilage, periosteum, heart tissue, spinal cord tissue, pancreatic tissue, kidney tissue, liver tissue, eye tissue, brain tissue, bladder tissue, prostate tissue, breast tissue, intestinal tissue, skeletal muscle tissue, lung tissue, a tendon, and a ligament; or
(v) the target region is a wound or lesion in a tissue of the patient selected from the group consisting of bone tissue, articular cartilage, periosteum, heart tissue, spinal cord tissue, pancreatic tissue, kidney tissue, liver tissue, eye tissue, brain tissue, bladder tissue, prostate tissue, breast tissue, intestinal tissue, skeletal muscle tissue, lung tissue, a tendon, and a ligament and, wherein in the target region, articular cartilage is thinned or denuded, or has a defect.

3-6. (canceled)

7. The method of claim 1, wherein:
(i) the magnetic particles or magnetically inducible particles are implanted in subchondral bone of a joint surface;
(ii) the magnetic particles or magnetically inducible particles are implanted in subchondral bone of a joint surface and wherein the joint surface is a knee joint surface or a hip joint surface; or
(iii) the magnetic particles or magnetically inducible particles are implanted in subchondral bone of a joint surface and wherein the joint surface is a knee joint surface or a hip joint surface and wherein the knee joint surface is the surface of a femoral condyle.

8-9. (canceled)

10. The method of claim 1, wherein:
(i) the magnetic particles or magnetically inducible particles are implanted within 3 mm of the target region;
(ii) the target region is a wound or lesion in a tissue of the patient selected from the group consisting of bone tissue, articular cartilage, periosteum, heart tissue, spinal cord tissue, pancreatic tissue, kidney tissue, liver tissue, eye tissue, brain tissue, bladder tissue, prostate tissue, breast tissue, intestinal tissue, skeletal muscle tissue, lung tissue, a tendon, and a ligament and wherein the magnetic particles or magnetically inducible particles have an average diameter that is less than 50% of a maximum dimension of the region;
(iii) the magnetic particles or magnetically inducible particles have an average diameter from about 1 μm to about 500 μm;
(iv) the magnetic particles or magnetically inducible particles are implanted at a coverage ratio from about 10 to about 1000 particles per square centimeter of area of the target region;
(v) the magnetic particles or magnetically inducible particles, when magnetically induced, have an average magnetization from about 1 to about 10,000 amperes/meter (A/m)
(vi) the magnetic particles or magnetically inducible particles comprise a biodegradable or bone-compatible polymer
(vii) the magnetic particles or magnetically inducible particles comprise a biodegradable or bone-compatible polymer and wherein the bone-compatible polymer is selected from the group consisting of poly(methyl methacrylate) (PMMA), poly(lactic acid) (PLA), polyglycolide, polycaprolactone, poly(lactic-co-glycolic acid) (PGLA), ultra high molecular weight polyethylene (UHMWPE), polyhydroxybutyrate, a polyimminocarbonate, a polyanhydride, a polyethylene glycol/polybutylene terephthalate (PEG/PBT) copolymer, and a polyetherurathane;
(viii) the magnetic particles comprise a metal, or a compound of a metal, selected from the group consisting of iron, cobalt, nickel, and manganese;
(ix) the magnetic particles comprise a metal, or a compound of a metal, selected from the group consisting of iron, cobalt, nickel, and manganese and wherein the magnetic particles comprise magnetite or maghemite;
(x) magnetic particles comprise a magnetic polymer; or
(xi) the magnetically inducible particles are in the form of coils, aligned to induce a magnetic field through the target region when an electric current is established in the coils.

11-14. (canceled)

15. The method of claim 1, wherein:
(i) the magnetizable substrate comprises a ferromagnetic material, a ferrimagnetic material, or a paramagnetic material
(ii) the magnetizable substrate comprises a ferromagnetic material, a ferrimagnetic material, or a paramagnetic material and wherein the magnetizable substrate comprises a metal, a metal alloy, or a metal compound; or
(iii) the magnetizable substrate comprises a ferromagnetic material, a ferrimagnetic material, or a paramagnetic material and wherein the ferromagnetic material, ferrimagnetic material, or paramagnetic material is present as a coating layer on an underlying polymer base.

16-17. (canceled)

18. The method of claim 1, wherein the regenerative cells are complexed with the magnetizable substrate through an antigen-antibody binding interaction or a cell adhesion peptide interaction.

19-24. (canceled)

25. The method of claim 1, comprising, in step (a) implanting a plurality of magnetically inducible particles in the patient, wherein the magnetically inducible particles are in the form of coils, the method further comprising:
(c1) inducing a magnetic field in the target region by establishing an electric current in the coils; or
(c2) inducing a magnetic field in the target region by establishing an electric current in the coils, wherein step (c1) is performed temporarily to initiate engraftment of the stem cells.

26. The method of claim 25, wherein:
(i) the electric current in the coils is established with an external magnetic field; or
(ii) the electric current in the coils is established with an external magnetic field and wherein the electric current in the coils is variable, whereby the magnetic field induced by the coils is also variable.

27-28. (canceled)

29. The method of claim 1, further comprising, prior to steps (a) and (b),
(1a) preparing the regenerative cells complexed with a magnetizable substrate, from a tissue sample of the patient that contains the regenerative cells;
(1b) preparing the regenerative cells complexed with a magnetizable substrate, from a tissue sample of the patient that contains the regenerative cells, wherein the tissue sample is a blood or bone marrow sample of the patient;
(1c) preparing the regenerative cells complexed with a magnetizable substrate, from a tissue sample of the patient that contains the regenerative cells, wherein one or more cell surface recognition ligands are bound to the magnetizable substrate and step (I) comprises selectively complexing stem cells of a desired phenotype with the magnetizable substrate, through an interaction between the cell surface recognition ligands and a surface marker of the desired phenotype; or
(1d) preparing the regenerative cells complexed with a magnetizable substrate, from a tissue sample of the patient that contains the regenerative cells, wherein one or more cell surface recognition ligands are bound to the magnetizable substrate and step (I) comprises selectively complexing stem cells of a desired phenotype with the magnetizable substrate, through an interaction between the cell surface recognition ligands and a surface marker of the desired phenotype, wherein the stem cells of the desired phenotype are mesenchymal stem cells (MSCs) and the interaction is an antigen-antibody binding interaction.

30-32. (canceled)

33. A kit for promoting wound repair with regenerative cells in a patient, the kit comprising:
(a) ferromagnetic beads having cell surface recognition ligands bonded thereto;
(b) magnetic spheres having an average diameter of less than about 1 mm and a magnetization of less than about 10,000 amperes/meter; and
(c) an elongated implantation device adapted to implant the magnetic spheres in a tissue of the patient
wherein (a), (b), and (c) are packaged together.

34. The kit of claim 33, wherein:
(i) the cell surface recognition ligands are anti-CD44 antibodies
(ii) wherein the elongated implantation device is adapted for implanting the magnetic spheres into subchondral bone of the patient;
(iii) the kit further comprises magnetically inducible coils, at least a portion of which have an average coil diameter of less than about 5 mm;
(iv) the kit further comprises magnetically inducible coils, at least a portion of which have an average coil diameter of less than about 5 mm and a magnetic field generating article or apparatus adapted to externally induce a magnetic field through the coils when implanted in the patient; or
(v) the kit further comprises magnetically inducible coils, at least a portion of which have an average coil diameter of less than about 5 mm and a magnetic field generating article or apparatus adapted to externally induce a magnetic field through the coils when implanted in the patient and wherein the magnetic field generating article is further adapted to fit over, or be worn over, a knee or hip joint of the patient.

35-38. (canceled)

39. A method for regenerating articular cartilage in a patient, the method comprising:
(a1) magnetically isolating mesenchymal stem cells (MSCs) proximate an articular cartilage lesion, using a plurality of magnetic spheres implanted in the patient in a three-dimensional profile that is specific to the geometry of the articular cartilage lesion; or
(a2) magnetically isolating mesenchymal stem cells (MSCs) proximate an articular cartilage lesion, using a plurality of magnetic spheres implanted in the patient in a three-dimensional profile that is specific to the geometry of the articular cartilage lesion, wherein the articular cartilage is hyaline cartilage.

40. (canceled)

41. A composition of matter, which is:
(A) a magnetic sphere having:
(i) an average diameter of less than about 1 mm and adapted to be implanted in a subchondral bone of a patient, wherein the magnetic sphere comprises a bone-compatible polymer; or
(ii) an average diameter of less than about 1 mm and adapted to be implanted in a subchondral bone of a patient, wherein the magnetic sphere comprises a bone-compatible polymer, wherein the magnetic sphere comprises a magnetic polymer; or
(B) a magnetically-inducible coil having:
a coil diameter of less than about 5 mm and adapted to be implanted in a subchondral bone of a patient and to generate a magnetic field in a target region within the patient, upon exposure to an externally induced magnetic field.

42-43. (canceled)
